(19) United States
(12) Patent Application Publication
(10) Pub. No.: US 2026/0345913 A1
(43) Pub. Date: Nov. 5, 2026

(54) PLUNGER ASSEMBLIES FOR SEALING PHARMACEUTICAL CONTAINERS AT LOW TEMPERATURES

(71) Applicant: CORNING INCORPORATED, CORNING, NY (US)

(72) Inventors: Dane Alphanso Christie, Painted Post, NY (US); Harikrishnan Rajendran, Madison, AL (US); Adam Robert Sarafian, Corning, NY (US); Jiangtao Wu, Corning, NY (US); Jin Wu, Pittsford, NY (US)

(21) Appl. No.: 19/486,694

(22) PCT Filed: May 17, 2024
(86) PCT No.: PCT/US2024/029819

§ 371 Date: Nov. 21, 2025

Related U.S. Application Data
(60) Provisional application No. 63/469,955, filed on May 31, 2023.

(51) Int. Cl. A61M 5/315; B65B 3/00

(52) U.S. Cl. CPC A61M 5/31513; B65B 3/003; B65B 2220/24

(57) ABSTRACT

Disclosed herein are plunger assemblies for sealing pharmaceutical containers. According to one or more embodiments described herein a plunger assembly may include a plunger comprising a resilient material having a glass transition temperature T_{g} of less than or equal to −65° C. and a plunger rod coupled to the plunger. Also disclosed herein are pharmaceutical container assemblies and methods of storing pharmaceutical compositions.

### CROSS-REFERENCE TO RELATED APPLICATIONS

This application claims the benefit of priority under 35 U.S.C. § 119 of U.S. Provisional Application Ser. No. 63/469,955 filed on May 31, 2023, the content of which is relied upon and incorporated herein by reference in its entirety.

### TECHNICAL FIELD

The present specification generally relates to pharmaceutical containers, and more particularly, to plunger assemblies for sealing pharmaceutical containers.

### BACKGROUND

Historically, glass has been used to produce a variety of articles. In particular, because of its hermeticity, optical clarity, and excellent chemical durability relative to other materials, glass has been a preferred material for pharmaceutical applications, including, without limitation, vials, syringes, ampoules, cartridges, jars, and other glass articles. These pharmaceutical containers may be sealed with plungers, stoppers, or other closures to preserve the integrity of the materials stored within the containers.

### SUMMARY

Low storage temperatures may result in dimensional changes in the closure components (e.g., the glass or plastic container, and/or the plunger), leading to issues in the integrity of the seal, and potential contamination of the material stored therein. Accordingly, an ongoing need exists for pharmaceutical containers suitable for use at low temperatures and in particular for plunger assemblies for sealing pharmaceutical containers at low temperatures. The present disclosure is directed to plunger assemblies that can be used to seal a pharmaceutical container at low temperatures and to pharmaceutical container assemblies utilizing such plunger assemblies.

According to one or more embodiments, a plunger assembly for sealing a pharmaceutical container may comprise a plunger which may comprise a resilient material that may have a glass transition temperature T_{g} of less than or equal to −65° C. The plunger assembly may also comprise a plunger rod coupled to the plunger.

According to additional embodiments, a pharmaceutical container assembly may comprise a pharmaceutical container that may comprise a body having an outer surface and an inner surface and a plunger that may be inserted within an opening of the body. An outer radial surface of the plunger may form an interference fit with the inner surface of the body. The plunger may comprise a resilient material that may have a glass transition temperature T_{g} of less than or equal to −65° C. The interference fit may form a seal between the inner surface of the body of the pharmaceutical container and the outer surface of the plunger. The seal may be maintained when the pharmaceutical container assembly is cooled to less than or equal to −65° C.

According to additional embodiments, a method of storing a pharmaceutical composition may comprise providing a pharmaceutical container assembly. The pharmaceutical container assembly may comprise a body having an outer surface and an inner surface and a plunger that may be inserted within an opening of the body. An outer radial surface of the plunger may form an interference fit with the inner surface of the body. The interference fit may form a seal between the inner surface of the body of the pharmaceutical container and the outer surface of the plunger. The plunger may comprise a resilient material that may have a glass transition temperature of less than or equal to −65° C. The method may also comprise adding a biological or pharmaceutical composition to the pharmaceutical container. The method may further comprise inserting the plunger into the pharmaceutical container and cooling the pharmaceutical container assembly and the biological or pharmaceutical composition disposed therein to a temperature of less than −65° C. The seal may be maintained when the pharmaceutical container assembly is cooled to less than or equal to −65° C.

Additional features and advantages of the embodiments described herein will be set forth in the detailed description which follows, and in part will be readily apparent to those skilled in the art from that description or recognized by practicing the embodiments described herein, including the detailed description which follows, the claims as well as the appended drawings.

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate the various embodiments described herein, and together with the description serve to explain the principles and operations of the claimed subject matter.

### BRIEF DESCRIPTION OF THE DRAWINGS

FIG. 1A schematically depicts a cross-section of a plunger assembly according to one or more embodiments shown and described herein;

FIG. 1B schematically depicts a cross-section of a plunger assembly according to one or more embodiments shown and described herein;

FIG. 2 schematically depicts a cross-section of a pharmaceutical container assembly, according to one or more embodiments shown and described herein;

FIG. 3A depicts simulated contact pressure at 25° C. between a pharmaceutical container and a plunger assembly having a glass transition temperature of −60° C., according to one or more embodiments shown and described herein;

FIG. 3B graphically depicts an interface between the plunger assembly and the pharmaceutical container in the simulation of FIG. 3A, where the differences in seal pressure are annotated using differences in shading patterns, according to one or more embodiments shown and described herein;

FIG. 4 graphically depicts contact area (y-axis) as a function of temperature (x-axis) for three different cooling rates for a pharmaceutical container assembly comprising a pharmaceutical container and a plunger assembly (glass transition temperature of −60° C.) engaged at an interference fit of 5%, according to one or more embodiments shown and described herein;

FIG. 5 graphically depicts contact area (y-axis) as a function of temperature (x-axis) for three different cooling rates for a pharmaceutical container assembly comprising a pharmaceutical container and a plunger assembly (glass transition temperature of −60° C.) engaged at an interference fit of 10%, according to one or more embodiments shown and described herein;

FIG. 6 graphically depicts contact area (y-axis) as a function of temperature (x-axis) for five different cooling rates for a pharmaceutical container assembly comprising a pharmaceutical container and a plunger assembly (glass transition temperature of −60° C.) engaged at an interference fit of 5%, according to one or more embodiments shown and described herein;

FIG. 7A depicts simulated contact pressure at 25° C. between a pharmaceutical container and a plunger assembly having a glass transition temperature of −130° C., according to one or more embodiments shown and described herein;

FIG. 7B graphically depicts an interface between the plunger assembly and the pharmaceutical container in the simulation of FIG. 7A, where the differences in seal pressure are annotated using differences in shading patterns, according to one or more embodiments shown and described herein;

FIG. 8A depicts simulated contact pressure at −80° C. between a pharmaceutical container and a plunger assembly having a glass transition temperature of −130° C., according to one or more embodiments shown and described herein;

FIG. 8B graphically depicts an interface between the plunger assembly and the pharmaceutical container in the simulation of FIG. 8A, where the differences in seal pressure are annotated using differences in shading patterns, according to one or more embodiments shown and described herein;

FIG. 9A depicts simulated contact pressure at −125° C. between a pharmaceutical container and a plunger assembly having a glass transition temperature of −130° C., according to one or more embodiments shown and described herein;

FIG. 9B graphically depicts an interface between the plunger assembly and the pharmaceutical container in the simulation of FIG. 9A, where the differences in seal pressure are annotated using differences in shading patterns, according to one or more embodiments shown and described herein; FIG. 10 graphically depicts contact area (y-axis) as a function of temperature (x-axis) for five different interference fits for a pharmaceutical container assembly comprising a pharmaceutical container and a plunger assembly (glass transition temperature of −130° C.) according to one or more embodiments shown and described herein;

FIG. 11A depicts the simulated contact pressure between a pharmaceutical container and a plunger assembly having a glass transition temperature of −130° C. and a butyl rubber coating layer at 25° C., according to one or more embodiments shown and described herein;

FIG. 11B graphically depicts an interface between the plunger assembly and the pharmaceutical container in the simulation of FIG. 11A, where the differences in seal pressure are annotated using differences in shading patterns, according to one or more embodiments shown and described herein;

FIG. 12A depicts the simulated contact pressure between a pharmaceutical container and a plunger assembly having a glass transition temperature of −130° C. and a butyl rubber coating layer at −80° C., according to one or more embodiments shown and described herein;

FIG. 12B graphically depicts an interface between the plunger assembly and the pharmaceutical container in the simulation of FIG. 12A, where the differences in seal pressure are annotated using differences in shading patterns, according to one or more embodiments shown and described herein;

FIG. 13A depicts the simulated contact pressure between a pharmaceutical container and a plunger assembly having a glass transition temperature of −130° C. and a butyl rubber coating layer at −130° C., according to one or more embodiments shown and described herein; and

FIG. 13B graphically depicts an interface between the plunger assembly and the pharmaceutical container in the simulation of FIG. 13A, where the differences in seal pressure are annotated using differences in shading patterns, according to one or more embodiments shown and described herein.

### DETAILED DESCRIPTION

Embodiments of the present application will now be described, examples of which are illustrated in the accompanying drawings. Whenever possible, the same reference numerals will be used throughout the drawings to refer to the same or like parts. The present disclosure may, however, be embodied in different forms and should not be construed as limited to the embodiments set forth in this disclosure. Rather, these embodiments are provided so that this disclosure will be thorough and complete, and will fully convey the scope of the subject matter to those skilled in the art.

Embodiments of the present disclosure are directed to plunger assemblies for sealing a pharmaceutical container. The plunger assembly may comprise a plunger comprising a resilient material having a glass transition temperature T_{g} of less than or equal to −65° C. and a plunger rod coupled to the plunger. The plunger assemblies disclosed herein may be utilized in a pharmaceutical container assembly. The pharmaceutical container assembly may comprise a pharmaceutical container comprising a body having an outer surface and an inner surface. The plunger assembly may be inserted within an opening within the body. An outer radial surface of the plunger may form an interference fit with the inner surface of the body. The interference fit may form a seal between the inner surface of the body of the pharmaceutical container and the outer surface of the plunger. The seal may be maintained when the pharmaceutical container is cooled to less than −65° C.

As used herein, the term “container closure integrity” refers to maintenance of a seal at an interface between a pharmaceutical container and a sealing assembly (e.g., between a sealing surface of a pharmaceutical container and a plunger) that is free of gaps above a threshold size to maintain a probability of contaminant ingress or reduce the possibility of gas permeability below a predetermined threshold based on the material stored in a pharmaceutical container. For example, in embodiments, a container closure integrity is maintained if a helium leakage rate during a helium leak test described in USP<1207> (2016) is maintained at less than or equal to 1.4×10⁻⁶ cm³/s.

As used herein, the term “CTE,” refers to the coefficient of linear thermal expansion of a material at a temperature of 25° C., unless stated otherwise.

As used herein, the term “resilient material,” refers to a material that when deformed, such as by compression, has the ability to return to its original shape.

As used herein, the term “biocompatible polymer,” refers to a polymer that does not react with or negatively affect a pharmaceutical or biological composition when contacted by the composition.

As used herein, the term “interference fit,” refers to a form of fastening between two parts that produces a joint held together by friction after the parts are pushed together.

As used herein, the term “interference fit percent,” refers to the percent difference between the diameter of the outer radial surface of the plunger and the diameter of the interior surface of the body of the pharmaceutical container.

Pharmaceutical containers, such as syringes, are typically sealed via a plunger or other closure to preserve the integrity of the contained material. Closures, such as plungers are typically made of synthetic rubbers and other elastomers. The closures are may be held in place by the interference fit between the closure and the pharmaceutical container. Some biological materials (e.g., blood, serum, proteins, stem cells, and other perishable biological fluids) require storage at low temperatures, such as temperatures less than −45° C., less than −80° C., or even less than about −180° C. For example, certain RNA-based vaccines may require storage at dry-ice temperatures (e.g., approximately −80° C.) or liquid nitrogen temperatures (e.g., approximately −180° C.) to remain active.

The plunger assemblies, pharmaceutical container assemblies, and methods of storing pharmaceutical compositions of the present disclosure may be utilized at temperatures less than those of conventional plunger or container assemblies, such as at a temperature less than or equal to −65° C. Typically, plunger assemblies used at low temperatures may not sufficiently seal a pharmaceutical container, as the glass transition temperature of the materials used to form conventional plungers may be above the temperatures at which the pharmaceutical container is stored. Without being bound by any particular theory, it is believed that loss of seal integrity at temperatures less than or equal to −65° C. may be caused by differences in thermal shrinkage between various components, loss of resiliency of the plunger at temperatures less than the glass transition temperature of the material from which the plunger is made, or a combination of these.

The plunger assemblies, pharmaceutical container assemblies and methods of storing pharmaceutical compositions of the present disclosure may allow for the use of lower storage temperatures, such as temperatures less than −65° C., which may be required to store certain pharmaceutical and/or biological compositions.

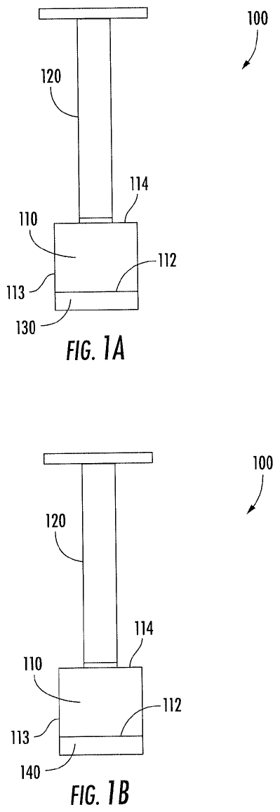

Referring now to FIG. 1A, one embodiment of a plunger assembly 100 for sealing a pharmaceutical container is schematically depicted. The plunger assembly 100, may comprise a plunger 110 and a plunger rod 120 coupled to the plunger. The plunger 110 may comprise an inner axial surface 112 and an outer axial surface 114. In embodiments, the plunger rod 120 may be coupled to the outer axial surface 114, and the inner axial surface 112 may face towards an interior of a pharmaceutical container when the plunger assembly is inserted into the pharmaceutical container. The plunger 110 may also have an outer radial surface 113 facing radially outward In embodiments, the plunger may comprise a resilient material that has a glass transition temperature T_{g} of less than or equal to −65° C. Below the T_{g}, the resilient material may behave as a solid (e.g., loss its elasticity), resulting in a diminished sealing force. That is, the resilient material effectively behaves as two different materials as it is cooled below its glass transition temperature: an elastic material above the transition temperature, and a solid glass below the transition temperature. In embodiments, the resilient material may have a T_{g} of less than or equal to −70° C., less than or equal to −80° C., less than or equal to −90° C., less than or equal to −100° C., less than or equal to −110° C., or even less than or equal to −120° C. In embodiments, the resilient material may have a T_{g} of from −65° C. to −150° C., such as from −65° C. to −140° C., from −65° C. to −130° C., from 65° C. to −120° C., from −65° C. to −110° C., from −65° C. to −100° C., from −65° C. to −90° C., from −65° C. to −80° C., from −65° C. to −70° C., from −70° C. to −150° C., from −70° C. to −140° C., from −70° C. to −130° C., from −70° C. to −120° C., from −70° C. to −110° C., from −70° C. to −100° C., from −70° C. to −90° C., from −70° C. to −80° C., from −80° C. to −150° C., from −80° C. to −140° C., from −80° C. to −130° C., from −80° C. to −120° C., from −80° C. to −110° C., from −80° C. to −100° C., from −80° C. to −90° C., from −90° C. to −150° C., from −90° C. to −140° C., from −90° C. to −130° C., from −90° C. to −120° C., from −90° C. to −110° C., from −90° C. to −100° C., from −100° C. to −150° C., from 100° C. to −140° C., from −100° C. to −130° C., from −100° C. to −120, from −110° C. to −150° C., from 110° C. to −140° C., from −110° C. to −130° C., from −110° C. to −120° C., from −120° C. to −150° C., from −120° C. to −140° C., from −120° C. to −130° C., from −130° C. to −150° C., from −130 ° to −140° C., from −140° C. to −150° C. or any combination of one or more of these ranges. In embodiments, the resilient material may comprise a silicone rubber that has a glass transition temperature T_{g} of less than or equal to −65° C.

Without being bound by theory, if the glass transition temperature of the resilient material is less than the intended temperature of use, a resilient material may lose its resiliency and the loss of resiliency may cause a loss of integrity of the seal. Maintenance of the seal of the plunger assembly may be determined by the compression of the plunger and the resiliency of the plunger at temperatures above its glass transition temperature. Accordingly, the plunger assemblies of the disclosed herein may be utilized at temperatures less than those of conventional plunger assemblies, which may utilize resilient materials, such as butyl rubber, with glass transition temperatures greater than −65° C.

Pharmaceutical and biological compositions that may be stored in pharmaceutical containers may be sensitive to or react with the resilient materials used in the plunger assemblies. This may result in contamination of the compositions stored within pharmaceutical containers. Referring again to FIG. 1, in embodiments, the plunger assembly 100 may further comprise a biocompatible polymer. The biocompatible polymer may reduce or prevent interactions between the pharmaceutical compositions and the resilient material and reduce or prevent contamination of the pharmaceutical composition resulting therefrom. In embodiments, the biocompatible polymer may comprise one or more of butyl rubber, bromobutyl rubber, chlorobutyl rubber, polytetrafluoroethylene, ethylene tetrafluoroethylene, fluorinated ethylene propylene, perfluoroalkoxy alkane, copolymers thereof, and blends thereof. In embodiments, the biocompatible polymer may comprise a polymer selected from the group consisting of butyl rubber, bromobutyl rubber, chlorobutyl rubber, polytetrafluoroethylene, ethylene tetrafluoroethylene, fluorinated ethylene propylene, perfluoroalkoxy alkane, copolymers thereof, and combinations thereof.

In embodiments, the biocompatible polymer may form a coating layer 130 on the inner axial surface 112 of the plunger 110. In embodiments, the coating layer 130 may have a thickness of at least 3 microns, such as at least 5 microns, at least 10 microns, at least 20 microns, at least 30 microns, at least 40 microns, or even at least 50 microns. In embodiments, the coating layer 130 may have a thickness of from about 3 microns to about 200 microns, such as from about 3 microns to about 175 microns, from about 3 microns to about 150 microns, from about 3 microns to about 125 microns, from about 3 microns to about 100 microns, from about 3 microns to about 75 microns, from about 3 microns to about 50 microns, from about 3 microns to about 25 microns, from about 25 microns to about 200 microns, from about 25 microns to about 175 microns, from about 25 microns to about 150 microns, from about 25 microns to about 125 microns, from about 25 micron to about 100 microns, from about 25 microns to about 75 microns, from about 25 microns to about 50 microns, from about 50 microns to about 200 microns, from about 50 microns to about 175 microns, from about 50 microns to about 125 microns, from about 50 microns to about 100 microns, from about 50 microns to about 75 microns, from about 75 microns to about 200 microns, from about 75 microns to about 175 microns, from about 75 microns to about 125 microns, from about 75 microns to about 100 microns, from about 100 microns to about 200 microns, from about 100 microns to about 175 microns, from about 100 microns to about 150 microns, from about 100 microns to about 125 microns, from about 125 microns to about 200 microns, from about 125 microns to about 175 microns, from about 125 microns to about 150 microns, from about 150 microns to about 200 microns, from about 150 microns to about 175 microns, from about 175 microns to about 200 microns, or any combination of one or more of these ranges.

In embodiments, the coating layer 130 may have a thickness of from greater than 0% to less than or equal to 75 % of the total thickness of the plunger 110, such as from about 0% to about 70%, from about 0% to about 60 %, from about 0% to about 50 %, from about 0% to about 40%, from about 0% to about 30%, from about 0% to about 20%, from about 0% to about 10 %, from about 10% to about 75%, from about 10% to about 70%, from about 10% to about 60%, from about 10% to about 50%, from about 10% to about 40%, from about 10% to about 30%, from about 10% to about 20%, from about 20% to about 75%, from about 20% to about 70%, from about 20% to about 60%, from about 20% to about 50%, from about 20% to about 40%, from about 20% to about 30%, from about 30% to about 75%, from about 30% to about 70%, from about 30% to about 60%, from about 30% to about 50%, from about 30% to about 40%, from about 40% to about 75%, from about 40% to about 70%, from about 40% to about 60%, from about 40% to about 50%, from about 50% to about 75%, from about 50% to about 70%, from about 50% to about 60%, from about 60% to about 75%, from about 60% to about 70%, from about 70% to about 75%, or any combination of one or more of these ranges.

The coating layer 130 may be formed on the inner axial surface 112 of the plunger 110 through thermoforming, spray coating, dip coating, or other coating processes. In embodiments, the coating layer 130 may be thermoformed onto the plunger 110. In embodiments, the coating layer 130 may be spray coated onto the plunger 110 and then cured. In embodiments, the coating layer 130 may be photo-cured after being spray coated onto the plunger 110. In embodiments, the coating layer 130 may comprise a biocompatible polymer that is curable upon exposure to UV light, and photo-curing may comprise exposing the biocompatible polymer to UV light for a period of time sufficient to cure the polymer.

In embodiments, the biocompatible polymer may be interspersed within the resilient material. In embodiments, the biocompatible polymer may be interspersed within the resilient material throughout the entire plunger 110. In embodiments, the biocompatible polymer may be interspersed within the resilient material throughout only a portion of the plunger 110. As shown in FIG. 1B, in embodiments, the biocompatible polymer may be interspersed within the resilient material on the inner axial surface 112 of the plunger 110 forming an interspersed polymer layer 140. In embodiments, the interspersed polymer layer 140 may have a thickness of at least 3 microns, such as at least 5 microns, at least 10 microns, at least 20 microns, at least 30 microns, at least 40 microns, or even at least 50 microns. In embodiments, the interspersed polymer layer 140 may have a thickness of from about 3 microns to about 200 microns, such as from about 3 microns to about 175 microns, from about 3 microns to about 150 microns, from about 3 microns to about 125 microns, from about 3 microns to about 100 microns, from about 3 microns to about 75 microns, from about 3 microns to about 50 microns, from about 3 microns to about 25 microns, from about 25 microns to about 200 microns, from about 25 microns to about 175 microns, from about 25 microns to about 150 microns, from about 25 microns to about 125 microns, from about 25 micron to about 100 microns, from about 25 microns to about 75 microns, from about 25 microns to about 50 microns, from about 50 microns to about 200 microns, from about 50 microns to about 175 microns, from about 50 microns to about 125 microns, from about 50 microns to about 100 microns, from about 50 microns to about 75 microns, from about 75 microns to about 200 microns, from about 75 microns to about 175 microns, from about 75 microns to about 125 microns, from about 75 microns to about 100 microns, from about 100 microns to about 200 microns, from about 100 microns to about 175 microns, from about 100 microns to about 150 microns, from about 100 microns to about 125 microns, from about 125 microns to about 200 microns, from about 125 microns to about 175 microns, from about 125 microns to about 150 microns, from about 150 microns to about 200 microns, from about 150 microns to about 175 microns, from about 175 microns to about 200 microns, or any combination of one or more of these ranges.

In embodiments, the interspersed polymer layer 140 may have a thickness of from greater than 0% to less than or equal to 75 % of the total thickness of the plunger 110, such as from about 0% to about 70%, from about 0% to about 60%, from about 0% to about 50%, from about 0% to about 40%, from about 0% to about 30%, from about 0% to about 20%, from about 0% to about 10%, from about 10% to about 75%, from about 10% to about 70%, from about 10% to about 60%, from about 10% to about 50%, from about 10% to about 40%, from about 10% to about 30%, from about 10% to about 20%, from about 20% to about 75%, from about 20% to about 70%, from about 20% to about 60%, from about 20% to about 50%, from about 20% to about 40%, from about 20% to about 30%, from about 30% to about 75%, from about 30% to about 70%, from about 30% to about 60%, from about 30% to about 50%, from about 30% to about 40%, from about 40% to about 75%, from about 40% to about 70%, from about 40% to about 60%, from about 40% to about 50%, from about 50% to about 75%, from about 50% to about 70%, from about 50% to about 60%, from about 60% to about 75%, from about 60% to about 70%, from about 70% to about 75%, or any combination of one or more of these ranges.

In embodiments, the interspersed polymer layer 140 may comprise from greater than 0 wt. % to 100 wt. % of the biocompatible polymer based on the total weight of the interspersed polymer layer 140, such as from greater than 0 wt. % to 90 wt. %, from greater than 0 wt. % to 80 wt. %, from greater than 0 wt. % to 70 wt. %, from greater than 0 wt. % to 60 wt. %, from greater than 0 wt. % to 50 wt. %, from greater than 0 wt. % to 40 wt. %, from greater than 0 wt. % to 30 wt. %, from greater than 0 wt. % to 20 wt. %, from greater than 0 wt. % to 10 wt. %, from 10 wt. % to 100 wt. %, from 10 wt. % to 90 wt. %, from 10 wt. % to 80 wt. %, from 10 wt. % to 70 wt. %, from 10 wt. % to 60 wt. %, from 10 wt. % to 50 wt. %, from 10 wt. % to 40 wt. %, from 10 wt. % to 30 wt. %, from 10 wt. % to 20 wt. %, from 20 wt. % to 100 wt. %, from 20 wt. % to 90 wt. %, from 20 wt. % to 80 wt. %, from 20 wt. % to 70 wt. %, from 20 wt. % to 60 wt. %, from 20 wt. % to 50 wt. %, from 20 wt. % to 40 wt. %, from 20 wt. % to 30 wt. %, from 30 wt. % to 100 wt. %, from 30 wt. % to 90 wt. %, from 30 wt. % to 80 wt. %, from 30 wt. % to 70 wt. %, from 30 wt. % to 60 wt. %, from 30 wt. % to 50 wt. %, from 30 wt. % to 40 wt. %, from 40 wt. % to 100 wt. %, from 40 wt. % to 90 wt. %, from 40 wt. % to 80 wt. %, from 40 wt. % to 70 wt. %, from 40 wt. % to 60 wt. %, from 40 wt. % to 50 wt. %, from 50 wt. % to 100 wt. %, from 50 wt. % to 90 wt. %, from 50 wt. % to 80 wt. %, from 50 wt. % to 70 wt. %, from 50 wt. % to 60 wt. %, from 60 wt. % to 100 wt. %, from 60 wt. % to 90 wt. %, from 60 wt. % to 80 wt. %, from 60 wt. % to 70 wt. %, from 70 wt. % to 100 wt. %, from 70 wt. % to 90 wt. %, from 70 wt. % to 80 wt. %, from 80 wt. % to 100 wt. %, from 80 wt. % to 90 wt. %, from 90 wt. % to 100 wt. %, or any combination of one or more of these ranges.

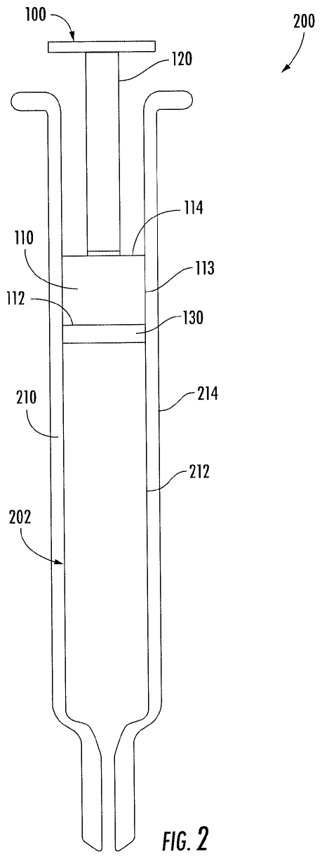

Now referring to FIG. 2, in embodiments, the plunger assembly 100 may be utilized in a pharmaceutical container assembly 200. The pharmaceutical container assembly 200 may comprise a pharmaceutical container 202 and the plunger assembly 100. The pharmaceutical container 202 may comprise a body 210, which may have an inner surface 212 and an outer surface 214. In embodiments, the pharmaceutical container 202 may be a syringe. While the pharmaceutical container assembly 200 is depicted in FIG. 2 as a syringe, it should be understood that the pharmaceutical container 202 may have other form factors, including, without limitation, Vacutainers®, cartridges, vials, bottles, flasks, phials, tubes, beakers or the like. The plunger assembly 100 may be inserted into an opening in the body 210. The plunger assembly 100 may have the outer radial surface 113 of the plunger 110, which is the surface of the plunger 110 that contacts the inner surface 212 of the body 210. The outer radial surface 113 of the plunger 110 may form an interference fit with the inner surface 212 of the body 210. In embodiments, the interference fit may form a seal between the inner surface 212 of the body 210 and the outer radial surface 113 of the plunger 110. In embodiments, the seal may be maintained when the pharmaceutical container assembly 200 is cooled to a temperature of less than or equal to −65° C.

In embodiments, the interference fit of the pharmaceutical container assembly 200 may be at least 2%, such as at least 3%, at least 4%, at least 5%, at least 6%, at least 7%, at least 8%, at least 9%, or even at least 10%. In embodiments, the interference fit of the pharmaceutical container assembly 200 may be from about 2% to about 10%, such as from about 2% to about 9%, from about 2% to about 8%, from about 2% to about 7%, from about 2% to about 6%, from about 2% to about 5%, from about 2% to about 4% from about 2% to about 3%, from about 3% to about 10%, from about 3% to about 9%, from about 3% to about 8%, from about 3% to about 7%, from about 3% to about 6%, from about 3% to about 5%, from about 3% to about 4%, from about 4% to about 10%, from about 4% to about 9%, from about 4% to about 8%, from about 4% to about 7%, from about 4% to about 6%, from about 4% to about 5%, from about 5% to about 10%, from about 5% to about 9%, from about 5% to about 8%, from about 5% to about 7%, from about 5% to about 6%, from about 6% to about 10%, from about 6% to about 9%, from about 6% to about 8%, from about 6% to about 7%, from about 7% to about 10%, from about 7% to about 9%, from about 7% to about 8%, from about 8% to about 10%, from about 8% to about 9%, from about 9% to about 10%, or any combination of one or more of these ranges. Without being bound by theory, it is believed that an interference fit of less than 2% may not provide sufficient container closure integrity even when the glass transition temperature of the resilient material is below the temperature at which the pharmaceutical container assembly is stored. It is believed that if difference between the CTE of the resilient material and the CTE of the pharmaceutical container assembly body is large enough the seal may be negatively affected regardless of the glass transition temperature of the of the resilient material.

In embodiments, the seal may be maintained when the pharmaceutical container assembly 200 is cooled to a temperature of less than or equal to −65° C., less than or equal to −70° C., less than or equal to −80° C., less than or equal to −90° C., less than or equal to −100° C., less than or equal to −110° C., or even less than or equal to −120° C. In embodiments, seal may be maintained when the pharmaceutical container assembly 200 is cooled to a temperature of from −65° C. to −150° C., such as from −65° C. to −140° C., from −65° C. to −130° C., from −65° C. to −120° C., from −65° C. to −110° C., from −65° C. to −100° C., from −65° C. to −90° C., from −65° C. to −80° C., from −65° C. to −70° C., from −70° C. to −150° C., from −70° C. to −140° C., from −70° C. to −130° C., from −70° C. to −120° C., from −70° C. to −110° C., from −70° C. to −100° C., from −70° C. to −90° C., from −70° C. to −80° C., from −80° C. to −150° C., from −80° C. to −140° C., from −80° C. to −130° C., from −80° C. to −120° C., from −80° C. to −110° C., from −80° C. to −100° C., from −80° C. to −90° C., from −90° C. to −150° C., from −90° C. to −140° C., from −90° C. to −130° C., from −90° C. to −120° C., from −90° C. to −110° C., from −90° C. to −100° C., from −100° C. to −150° C., from −100° C. to −140° C., from −100 ° C. to −130° C., from −100° C. to −120° C., from −100° C. to −110° C., from −110° C. to −150° C., from −110° C. to −140° C., from −110° C. to −130° C., from −110° C. to −120° C., from −120° C. to −150° C., from −120° C. to −140° C., from −120° C. to −130° C., from −130° C. to −150° C., from −130° C. to −140° C., from −140° C. to −150° C., or any combination of one or more of these ranges. Without being bound by theory, it is believed that the seal may be maintained at a temperature of less than or equal to −65° C. because the compression of the resilient material of the plunger 110 of the plunger assembly 100 by the inner surface 212 of the body 210 is maintained while cooled because the glass transition temperature of the resilient material of the plunger is less than or equal to −65° C. and thus the resiliency of the resilient material may be maintained while cooled.

In embodiments, a helium leakage rate of the sealed container may be less than or equal to 1.4×10⁻⁶ cm³/s during a helium leak test described in USP <1207> (2016), which is incorporated by reference herein in its entirety. A helium leakage rate of less than or equal to 1.4×10⁻⁶ cm³/s indicates the maintenance of a seal at the interface between the pharmaceutical container and the plunger assembly that is free of gaps above a threshold size to maintain a probability of contaminant ingress or reduce the possibility of gas permeability below a predetermined threshold based on the material stored in a pharmaceutical container.

In embodiments, the body 210 of the pharmaceutical container 202 may comprise glass or polymeric glass. In embodiments, the glass may be an aluminosilicate glass composition. In embodiments, the glass may be an aluminosilicate glass composition that meets Type 1 criteria as defined in USP <660>, such as those glass compositions disclosed in U.S. Pat. No. 8,551,898 hereby incorporated by reference in its entirety, and sold by Corning® Incorporated as Valor® Glass, and those disclosed in U.S. Pat. No. 9,145,329, hereby incorporated by reference in its entirety. In embodiments, the glass may be an alkali aluminosilicate glass such as those disclosed in U.S. Pat. No. 10,640,415, entitled Lithium Containing Aluminosilicate Glasses filed Nov. 29, 2017, hereby incorporated by reference in its entirety, or those disclosed in U.S. Patent Publication No. 2020/0290920, entitled Chemically Durable Aluminosilicate Glass Compositions and Glass Articles Formed Therefrom, filed Sep. 17, 2020, hereby incorporated by reference in its entirety. In embodiments, the glass may be an aluminosilicate glass composition that has been subjected to an etching process, such as acid etching or fluoride etching, to remove deposits on the inner surface 212 of the pharmaceutical container assembly 200. In embodiments, the glass may be a 33 expansion borosilicate glass such as those sold by DWK Life Sciences as KIMBAL® 33 or those sold by Schott as BOROFLOAT® 33. Expansion 33 glass has a coefficient of thermal expansion of 33 and is a Type 1A glass per USP <660>. In embodiments, the glass may be a 51 expansion borosilicate glass, such as those sold by DWK Life Sciences as KIMBAL® 51 or those sold by Corning® as 51-D clear borosilicate glass tubing. Expansion 51 glass has a coefficient of thermal expansion of 51 and is a Type 1B glass per USP <660>. In embodiments, where the glass is a 33 expansion or 51 expansion borosilicate glass the outer surface 214 of the pharmaceutical container 202 may be coated with an external coating for example suitable containers may be coated containers sold by Corning® Incorporated under the trademark Velocity®.

In embodiments, the glass may be a strengthened aluminosilicate glass. In embodiments, the strengthened aluminosilicate glass may be formed by ion-exchanging an aluminosilicate glass in a molten salt bath. The ion exchange process may be performed in an ion exchange medium under processing conditions such as, for example, those disclosed in U.S. Pat. No. 8,551,898 which is incorporated herein by reference in its entirety and those disclosed in U.S. Pat. No. 9,145,329 which is incorporated herein by reference in its entirety. However, it should be understood that the ion-exchange process is not particularly limited and that other processes are contemplated herein.

Referring again to FIG. 2, in embodiments, a method of storing a pharmaceutical composition may comprise providing a pharmaceutical container assembly 200. Pharmaceutical container assembly 200 may comprise the pharmaceutical container 202 and the plunger assembly 100. The pharmaceutical container 202 may comprise the body 210, which may have the inner surface 212 and an outer surface 214. The plunger assembly 100 may comprises the plunger 110, which may be inserted within an opening of the body 210. The plunger 110 may have the outer radial surface 113, which is the portion of the outer surface of the plunger 110 that contacts the inner surface 212 of the body 210. The outer radial surface 113 may form an interference fit with the inner surface 212. In embodiments, the interference fit may form a seal between the inner surface 212 and the outer axial surface 114. The method may further comprise adding a biological or pharmaceutical composition to the pharmaceutical container. The method may comprise inserting the plunger 110 into the pharmaceutical container 202 and cooling the pharmaceutical container assembly 200 and the biological or pharmaceutical composition deposited therein to storage a temperature of less than −65° C. The seal may be maintained when the pharmaceutical container assembly 200 is cooled to the storage temperature of less than or equal to −65° C. In embodiments, the biological or pharmaceutical composition is added to the pharmaceutical container before the plunger is inserted into the pharmaceutical composition. In embodiments, the biological or pharmaceutical composition is added to the pharmaceutical container after the plunger is inserted into the pharmaceutical container.

In embodiments, the seal may be maintained at a storage temperature of less than −70° C., less than −80° C., less than −90° C., less than −100° C., less than −110° C., less than −120° C., less than −130° C., less than −140° C., or even less than −150° C. In embodiments, the seal may be maintained at a storage temperature of from −65° C. to −150° C., such as from −65° C. to −140° C., from −65° C. to −130° C., from −65° C. to −120° C., from −65° C. to −110° C., from −65° C. to −100° C., from −65° C. to −90° C., from −65° C. to −80° C., from −65° C. to −70° C., from −70° C. to −150° C., −70° C. to −140° C., from −70° C. to −130° C., from −70° C. to −120° C., from −70° C. to −110° C., from −70° C. to −100° C., from −70° C. to −90° C., from −70° C. to −80° C., from −80° C. to −150° C., −80° C. to −140° C., from −80° C. to −130° C., from −80° C. to −120° C., from −80° C. to −110° C., from −80° C. to −100° C., from −80° C. to −90° C., from −90° C. to −150° C., −90° C. to −140° C., from −90° C. to −130° C., from −90° C. to −120° C., from −90° C. to −110° C., from −90° C. to −100° C., from −100° C. to −150° C., −100° C. to −140° C., from −100° C. to −130° C., from −100° C. to −120° C., from −100° C. to −110° C., from −110° C. to −150 ° C., −110° C. to −140° C., from −110° C. to −130° C., from −110° C. to −120° C., from −120° C. to −150° C., −120° C. to −140° C., from −120° C. to −130° C., from −130° C. to −150° C., −130° C. to −140° C., from −140° C. to −150° C., or any combination of one or more of these ranges.

In embodiments, the cooling of the pharmaceutical container assembly 200 may occur at a rate of greater than or equal to −1° C./minute, such as greater than or equal to −3° C./minute, greater than or equal to −5° C./minute, greater than or equal to −10° C./minute, greater than or equal to −20° C./minute, greater than or equal to −30° C./minute, greater than or equal to −40° C./minute, greater than or equal to −50° C./minute, greater than or equal to −60° C./minute, greater than or equal to −70° C./minute, greater than or equal to −80° C./minute, greater than or equal to −90° C./minute, or even greater than or equal to −100° C./minute. In embodiments the cooling of the pharmaceutical container assembly 200 may occur at a rate of from −1° C./minute to −100° C./minute, such as from −1° C./minute to −90° C./minute, from −1° C./minute to −80° C./minute, from −1° C./minute to −70° C./minute, from −1° C./minute to −60° C./minute, from −1° C./minute to −50° C./minute, from −1° C./minute to −40° C./minute, from −1° C./minute to −30° C./minute, from −1° C./minute to −20° C./minute, from −1° C./minute to −10° C./minute, from −1° C./minute to −5° C./minute, from −1° C./minute to −3° C./minute, from −3° C./minute to −100° C./minute, from −3° C./minute to −90° C./minute, from −3° C./minute to −80° C./minute, from −3° C./minute to −70° C./minute, from −3° C./minute to −60° C./minute, from−3° C./minute to −50° C./minute, from −3° C./minute to −40° C./minute, from −3° C./minute to −30° C./minute, from −3° C./minute to −20° C./minute, from −3° C./minute to −10° C./minute, from −3° C./minute to −5° C./minute, from −5° C./minute to −100° C./minute, from −5° C./minute to −90° C./minute, from −5° C./minute to −80° C./minute, from −5° C./minute to −70° C./minute, from −5° C./minute to −60° C./minute, from −5° C./minute to −50° C./minute, from −5° C./minute to −40° C./minute, from −5° C./minute to −30° C./minute, from −5° C./minute to −20° C./minute, from −5° C./minute to −10° C./minute, from −10° C./minute to −100° C./minute, from −10° C./minute to −90° C./minute, from −10° C./minute to −80° C./minute, from −10° C./minute to −70° C./minute, from −10° C./minute to −60° C./minute, from −10° C./minute to −50° C./minute, from −10° C./minute to −40° C./minute, from −10° C./minute to −30° C./minute, from −10° C./minute to −20° C./minute, from −20° C./minute to −100° C./minute, from −20° C./minute to −90° C./minute, from −20° C./minute to −80° C./minute, from −20° C./minute to −70° C./minute, from −20° C./minute to −60° C./minute, from −20° C./minute to −50° C./minute, from −20° C./minute to −40° C./minute, from −20° C./minute to −30° C./minute, from −30° C./minute to −100° C./minute, from −30° C./minute to −90° C./minute, from −30° C./minute to −80° C./minute, from −30° C./minute to −70° C./minute, from −30° C./minute to −60° C./minute, from −30° C./minute to −50° C./minute, from −30° C./minute to −40° C./minute, from −40° C./minute to −100° C./minute, from −40° C./minute to −90° C./minute, from −40° C./minute to −80° C./minute, from −40° C./minute to −70° C./minute, from −40° C./minute to −60° C./minute, from −40° C./minute to −50° C./minute, from −50° C./minute to −100° C./minute, from −50° C./minute to −90° C./minute, from −50° C./minute to −80° C./minute, from −50° C./minute to −70° C./minute, from −50° C./minute to −60° C./minute, from −60° C./minute to −100° C./minute, from −60° C./minute to −90° C./minute, from −60° C./minute to −80° C./minute, from −60° C./minute to −70° C./minute, from −70° C./minute to −100° C./minute, from −70° C./minute to −90° C./minute, from −70° C./minute to −80° C./minute, from −80° C./minute to −100° C./minute, from −80° C./minute to −90° C./minute, from −90° C./minute to −100° C./minute, or any combination of one or more of these ranges.

### EXAMPLES

Embodiments of the present application will be further clarified by the following examples. It should be understood that these examples are not limiting to the embodiments described above.

For the simulated contact pressure tests in the following examples the finite element analysis (FEA) software Abaqus was used for the simulations in all examples. Axisymmetric model was used for the model. The elastic material model was used for the glass container and viscoelastic material model was used for the rubber stopper of the plunger. Surface contact model between the stopper and the glass container was used and interference fit strain can be determined by the interference contact. The CTE change with temperature is considered in the model. The temperature of the whole system decreases from room temperature 25° C. to −130° C. The contact area between the glass container and the stopper can then be measured using the post-process of the Abaqus software.

### Comparative Example 1

### Effect of Cooling Rate and Interference Fit on Container Closure Integrity

In Comparative Example 1, the effect of cooling rate and interference fit on the container closure integrity of a pharmaceutical container assembly having a plunger assembly with a T_{g} of −60° C. is examined. The plunger for Comparative Example 1 comprises butyl rubber.

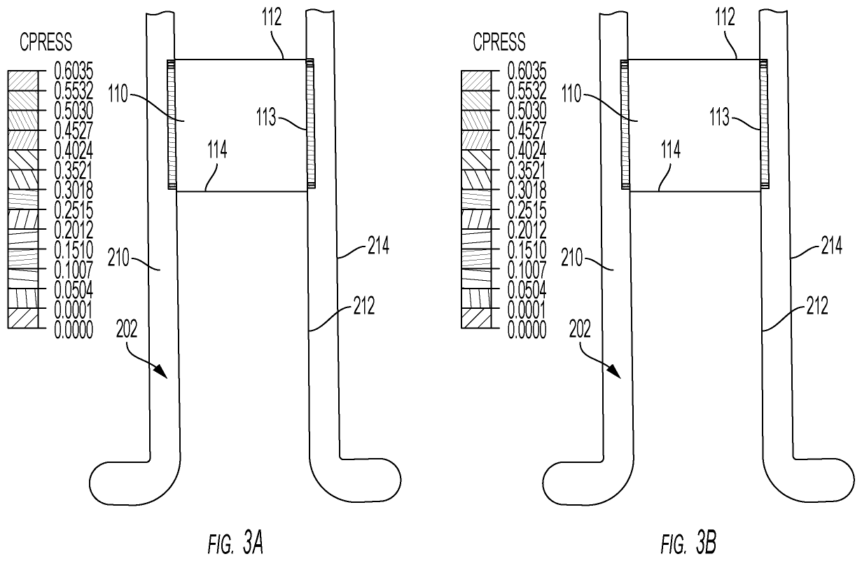

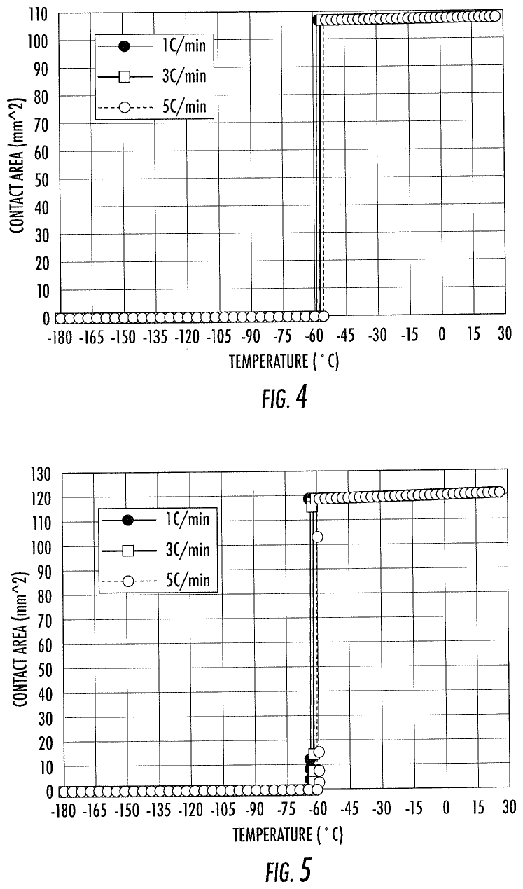

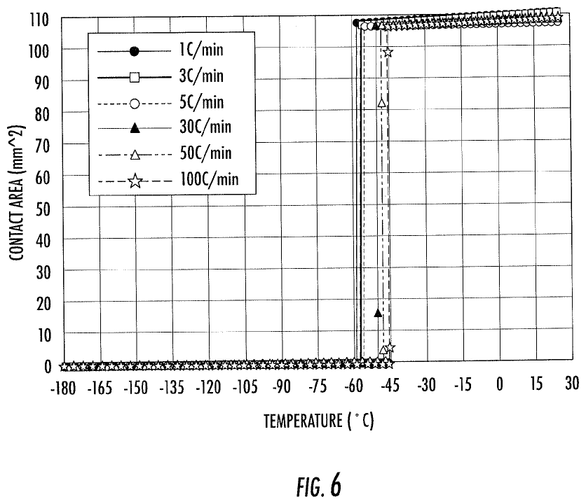

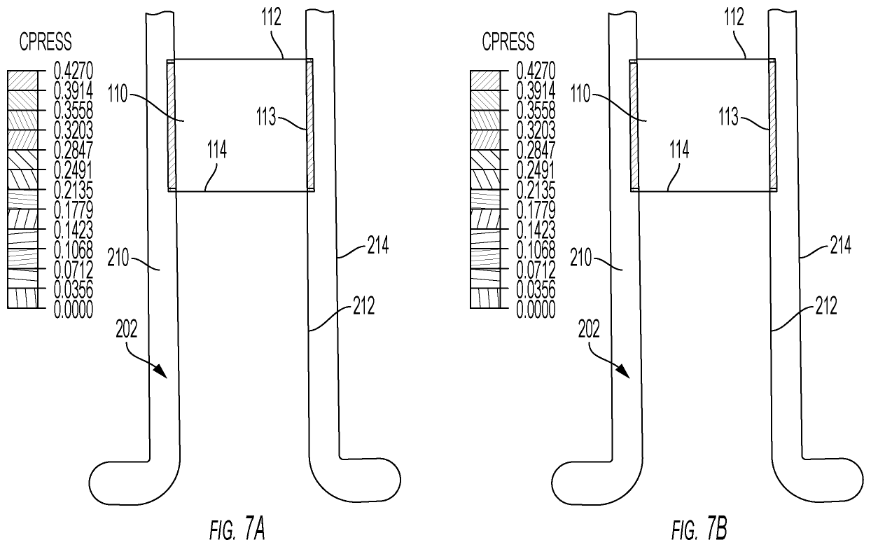

As shown in FIG. 3A and FIG. 3B the butyl rubber plunger exhibits good contact pressure at a temperature of 25° C. In FIGS. 4 and 5 the interference fit between the plunger assembly and the pharmaceutical container was adjusted between 5% and 10% respectively and the container assembly was cooled at 3 different cooling rates to determine the effect of cooling rate and interference fit percent on container closure integrity. The butyl rubber plunger has a T_{g} of approximately −60° C. and as shown in FIGS. 4 and 5 an increase in interference fit from 5% in FIG. 4 to 10% in FIG. 5 did not affect the failure temperature of the plunger. FIGS. 4 and 5 also show little impact on the failure rate from varying the cooling rates between −1° C./min, −2° C./min, and −3° C./min. Together FIGS. 4 and 5 demonstrate that the primary cause of failure is the glass transition temperature of the plunger. In FIG. 6 the effect of cooling rate on container closure integrity was further investigated at faster rates of cooling. For the simulation shown in FIG. 6 the simulated interference fit was 5% and the glass transition temperature of the plunger material was −60° C. FIG. 6 demonstrates that even more extreme cooling rates such as cooling rates of −30° C./min, −50°/min, and −100° C./min show only a small impact on the failure rate of the seal of the container, again indicating that the primary driver of failure is the glass transition temperature of the plunger.

### Example 2

### Effect of Glass Transition Temperature on Container Closure Integrity

In Example 2, the effect of the glass transition temperature on container closure integrity was examined by simulating a pharmaceutical container assembly containing a silicone rubber plunger with a glass transition temperature of approximately −130° C. The simulations were run at 25° C., −80° C., and −125° C. to determine container closure integrity at low temperature.

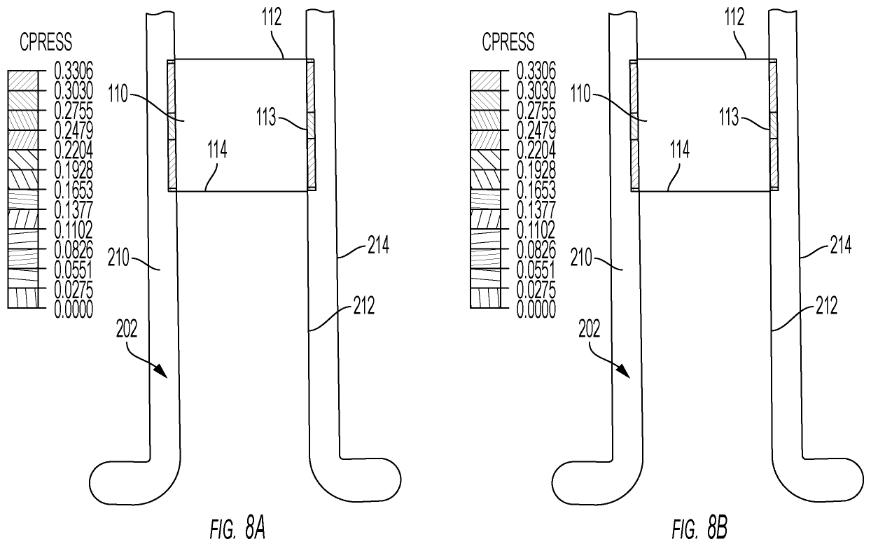

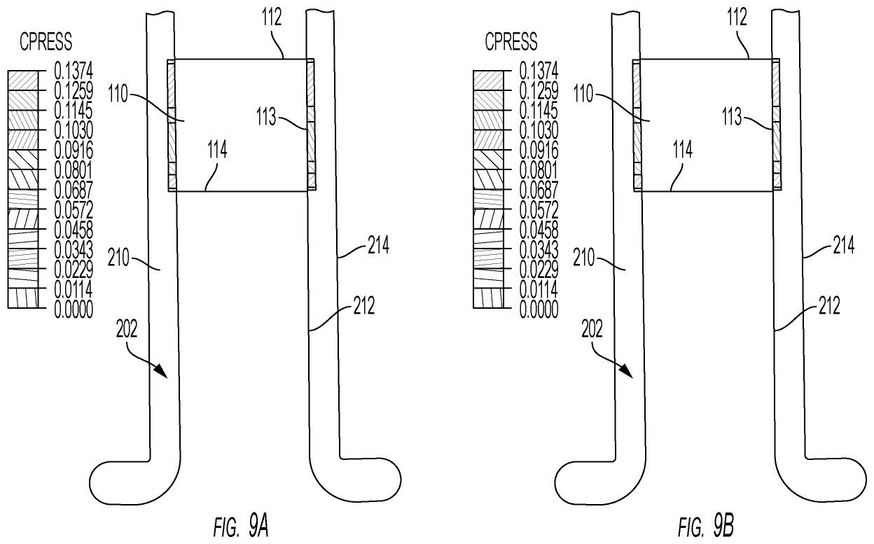

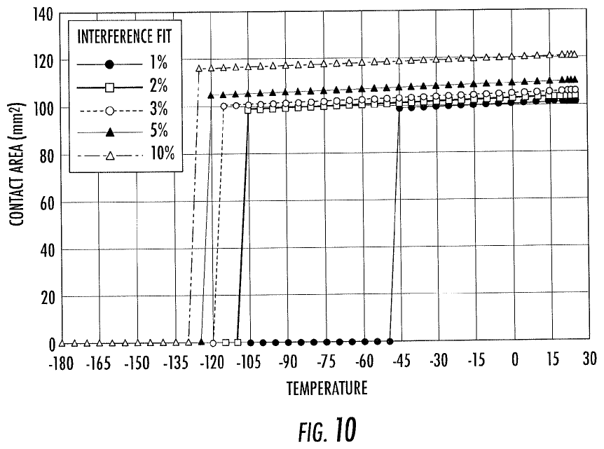

As shown in FIGS. 7A and 7B, the decreased glass transition temperature of the plunger did not negatively affect the performance of the plunger at 25° C. The contact pressure of the simulation shown in FIGS. 7A and 7B. Further, FIGS. 8A and 8B show that at −80° C. the plunger still demonstrated sufficient contact pressure to maintain container closure integrity indicating that decreasing the glass transition temperature of the plunger from −60° C. to −130° C. decreased the failure temperature of the pharmaceutical container assembly. FIGS. 9A and 9B, show that at a temperature of −125° C. the container closure integrity was still maintained.

### Example 3

### Effect of Interference Fit on Container Closure Integrity

In Example 3, the effect of interference fit on container closure integrity was examined by simulating a pharmaceutical container assembly containing a silicone rubber plunger with a glass transition temperature of approximately −130° C. at interference fit percentages of 1%, 2%, 3%, 5%, and 10%.

As shown in FIG. 10, at lower interference fit percentages, such as 1%, the interference fit increased the failure temperature well above the glass transition temperature of the plunger. While larger interference fit percentages did decrease the failure temperature, the effect of interference fit percentage on container closure integrity is most prominent at lower fit percentages and decreases as the interference fit percentage increases. FIG. 10 indicates that a minimum interference fit percentage exists to compensate for the shrinkage deformation caused by the CTE mismatch between the plunger and the pharmaceutical container, but increases beyond the minimum do not affect container closure integrity as significantly as the glass transition temperature of the plunger.

### Example 4

### Effect of a Biocompatible Polymer Coating on Container Closure Integrity

In Example 4, the effect of a biocompatible polymer coating on container closure integrity was examined by simulating a 200 micron thick coating of butyl rubber on a the inner surface of a silicone rubber plunger with a glass transition temperature of −130° C.

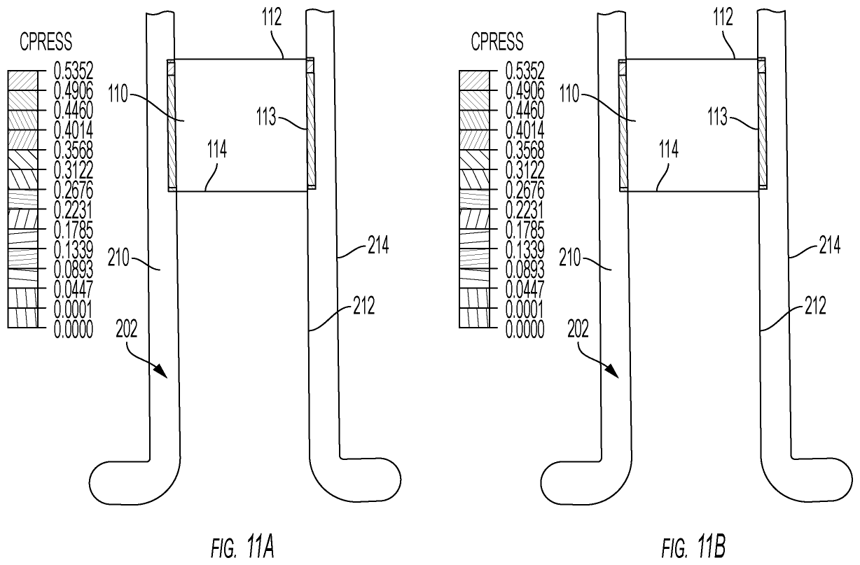

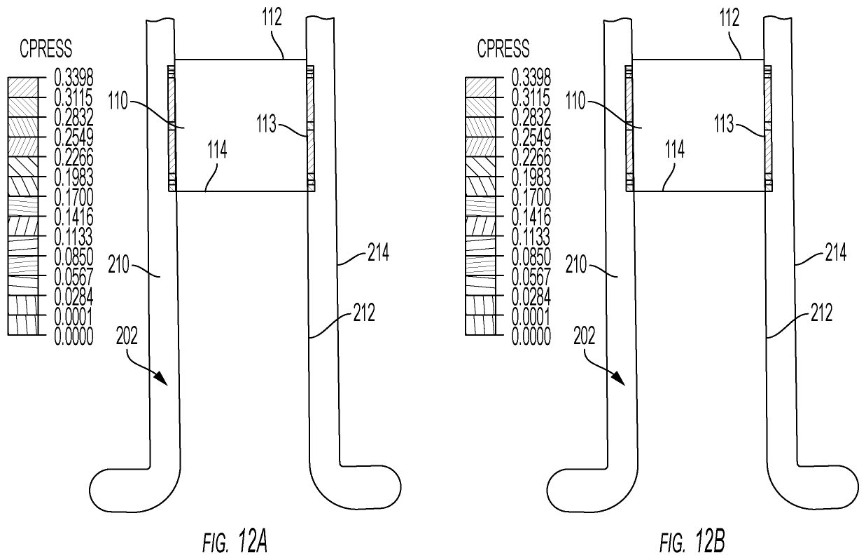

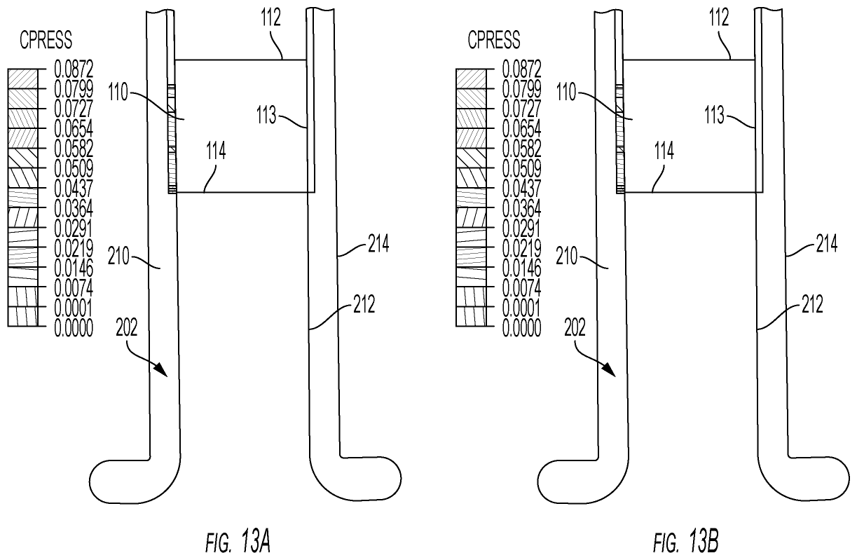

FIGS. 11A and 11B show the simulated contact pressure at 25° C. FIGS. 12A and 12B show the simulated contact pressure at −80° C. FIGS. 13A and 13B shows the simulated contact pressure at −130° C. Together, FIGS. 11A-13B show that adding a butyl rubber coating to the plunger did not prevent the maintenance of container closure integrity at temperatures as low as −130° C. despite the coatings higher glass transition temperature (approximately −60° C.) than that of the silicone rubber plunger. As the seal was maintained at similar temperatures to the simulations of Example 2 which did not have a biocompatible polymer coating.

According to a first aspect of the present disclosure, a plunger assembly for sealing a pharmaceutical container may comprise a plunger which may comprise a resilient material that may have a glass transition temperature T_{g} of less than or equal to −65° C. The plunger assembly may also comprise a plunger rod coupled to the plunger.

A second aspect of the present disclosure may include the plunger assembly of the first aspect where the plunger comprises an inner surface and an outer surface, the plunger rod is coupled to the outer surface of the plunger, and the inner surface faces towards an interior of the pharmaceutical container when the plunger assembly is inserted into the pharmaceutical container.

A third aspect of the present disclosure may include the plunger assembly of any of the first or second aspects, further comprising a biocompatible polymer.

A fourth aspect of the present disclosure may include the plunger assembly of the third aspect, where the biocompatible polymer comprises one or more of butyl rubber, bromobutyl rubber, chlorobutyl rubber, polytetrafluoroethylene, ethylene tetrafluoroethylene, fluorinated ethylene propylene, perfluoroalkoxy alkane, and copolymers or blends thereof.

A fifth aspect of the present disclosure may include the plunger assembly of any of the third or fourth aspects, where the biocompatible polymer forms a coating layer on the inner surface of the plunger.

A sixth aspect of the present disclosure may include the plunger assembly of the fifth aspect, where the coating layer has a thickness of at least 3 microns.

A seventh aspect of the present disclosure may include the plunger assembly of any of the fifth or sixth aspects, where the coating layer has a thickness of from greater than 0% to less than or equal to 75% of the total thickness of the plunger.

An eighth aspect of the present disclosure may include the plunger assembly of any of the fifth or sixth aspects, where the coating layer has a thickness of from about 25 microns to about 200 microns.

A ninth aspect of the present disclosure may include the plunger assembly of any of the fifth through eighth aspects, where the coating layer is thermoformed onto the plunger.

A tenth aspect of the present disclosure may include the plunger assembly of any of the fifth through eighth aspects, where the coating layer is spray coated onto the plunger.

An eleventh aspect of the present disclosure may include the plunger assembly of the tenth aspect, where the coating is photo −cured after being spray coated onto the plunger.

A twelfth aspect of the present disclosure may include the plunger assembly of any of the third or fourth aspects, where the biocompatible polymer is interspersed within the resilient material.

A thirteenth aspect of the present disclosure may include the plunger assembly of the twelfth aspect, where the biocompatible polymer is interspersed within the resilient material on the inner surface of the plunger forming an interspersed polymer layer.

A fourteenth aspect of the present disclosure may include the plunger assembly of the thirteenth aspect, where the interspersed polymer layer comprises from greater than 0 wt. % to 100 wt. % of the biocompatible polymer based on the total weight of the interspersed polymer layer.

A fifteenth aspect of the present disclosure may include the plunger assembly of any preceding aspect, where the resilient material has a glass transition temperature T_{g} of less than or equal to −80° C.

A sixteenth aspect of the present disclosure may include the plunger assembly of any preceding aspect, where the resilient material has a glass transition temperature T_{g} of less than or equal to −120° C.

A seventeenth aspect of the present disclosure may include the plunger assembly of any preceding aspect, where the resilient material comprises a silicone rubber that has a glass transition temperature T_{g} of less than or equal to −65° C.

According to an eighteenth aspect of the present disclosure, a pharmaceutical container assembly may comprise a pharmaceutical container that may comprise a body having an outer surface and an inner surface and a plunger that may be inserted within an opening of the body. An outer radial surface of the plunger may form an interference fit with the inner surface of the body. The plunger may comprise a resilient material that may have a glass transition temperature T_{g} of less than or equal to −65° C. The interference fit may form a seal between the inner surface of the body of the pharmaceutical container and the outer surface of the plunger. The seal may be maintained when the pharmaceutical container assembly is cooled to less than or equal to −65° C.

A nineteenth aspect of the present disclosure may include the pharmaceutical container assembly of the eighteenth aspect, where a helium leakage rate of the sealed container is less than or equal to 1.4×10⁻⁶ cm³/s at a temperature of −65° C.

A twentieth aspect of the present disclosure may include the pharmaceutical container assembly of any of the eighteenth or nineteenth aspects, where the interference fit is at least 2%, wherein the interference fit percent is the percent difference between the diameter of the outer radial surface of the plunger and the diameter of the inner surface of the body.

A twenty-first aspect of the present disclosure may include the pharmaceutical container assembly of any of the eighteenth through twentieth aspects, where the interference fit is at least 5%.

A twenty-second aspect of the present disclosure may include the pharmaceutical container assembly of any of the eighteenth through twenty-first aspects, where the plunger further comprises a biocompatible polymer.

A twenty-third aspect of the present disclosure may include the pharmaceutical container assembly of the twenty-second aspect, where the biocompatible polymer comprises one or more of butyl rubber, bromobutyl rubber, chlorobutyl rubber, polytetrafluoroethylene, ethylene tetrafluoroethylene, fluorinated ethylene propylene, perfluoroalkoxy alkane, and copolymers or blends thereof.

A twenty-fourth aspect of the present disclosure may include the pharmaceutical container assembly of any of the twenty-second or twenty-third aspects, where the biocompatible polymer forms a coating layer on an inner surface of the plunger.

A twenty-fifth aspect of the present disclosure may include the pharmaceutical container assembly of the twenty-fourth aspect, where the coating layer has a thickness of at least 3 microns.

A twenty-sixth aspect of the present disclosure may include the pharmaceutical container assembly of any of the twenty-fourth or twenty-fifth aspects, where the coating layer has a thickness of from greater than 0% to less than or equal to 75% of the total thickness of the plunger.

A twenty-seventh aspect of the present disclosure may include the pharmaceutical container assembly of the any of the twenty-fourth or twenty-fifth aspects, where the coating layer has a thickness of from about 25 microns to about 200 microns.

A twenty-eighth aspect of the present disclosure may include the pharmaceutical container assembly of the any of the twenty-fourth through twenty-seventh aspects, where the coating layer is thermoformed onto the plunger.

A twenty-ninth aspect of the present disclosure may include the pharmaceutical container assembly of the any of the twenty-fourth through twenty-seventh aspects, where the coating layer is spray coated onto the plunger.

A thirtieth aspect of the present disclosure may include the pharmaceutical container assembly of the twenty-ninth aspect, where the coating is photo-cured after being spray coated onto the plunger.

A thirty-first aspect of the present disclosure may include the pharmaceutical container assembly of any of the twenty-second or twenty-third aspects, where the biocompatible polymer is interspersed within the resilient material.

A thirty-second aspect of the present disclosure may include the pharmaceutical container assembly of the thirty-first aspect, where the biocompatible polymer is interspersed within the resilient material.

A thirty-third aspect of the present disclosure may include the pharmaceutical container assembly of the thirty-second aspect, where the biocompatible polymer is interspersed within the resilient material on the inner surface of the plunger forming an interspersed polymer layer.

A thirty-fourth aspect of the present disclosure may include the pharmaceutical container assembly of any of the eighteenth to thirty-third aspects, where the resilient material has a glass transition temperature T_{g} of less than or equal to −80° C.

A thirty-fifth aspect of the present disclosure may include the pharmaceutical container assembly of any of the eighteenth to thirty-fourth aspects, where the resilient material has a glass transition temperature T_{g} of greater than or equal to −120° C.

A thirty-sixth aspect of the present disclosure may include the pharmaceutical container assembly of any of the eighteenth to thirty-fifth aspects, where the resilient material comprises a silicone rubber that has a glass transition temperature T_{g} of less than or equal to −65° C.

A thirty-seventh aspect of the present disclosure may include the pharmaceutical container assembly of any of the eighteenth to thirty-sixth aspects, where the body of the pharmaceutical container comprises glass of polymeric glass.

A thirty-eighth aspect of the present disclosure may include the pharmaceutical container assembly of any of the eighteenth to thirty-seventh aspects, where the seal is maintained when the container is cooled to less than or equal to −80° C.

A thirty-ninth aspect of the present disclosure may include the pharmaceutical container assembly of any of the eighteenth to thirty-eighth aspects, where the seal is maintained when the container is cooled to less than or equal to −120° C.

A fortieth aspect of the present disclosure may include the pharmaceutical container assembly of any of the eighteenth to thirty-nineteenth aspects, where the pharmaceutical container is a syringe.

A forty-first aspect of the present disclosure may include the pharmaceutical container assembly of any of the eighteenth to thirty-nineteenth aspects, where the pharmaceutical container is a cartridge.

According to a forty-second aspect of the present disclosure, a method of storing a pharmaceutical composition comprises providing a pharmaceutical container assembly. The pharmaceutical container assembly may comprise a body having an outer surface and an inner surface and a plunger that may be inserted within an opening of the body. An outer radial surface of the plunger may form an interference fit with the inner surface of the body. The interference fit may form a seal between the inner surface of the body of the pharmaceutical container and the outer surface of the plunger. The plunger may comprise a resilient material that may have a glass transition temperature of less than or equal to −65° C. The method may also comprise adding a biological or pharmaceutical composition to the pharmaceutical container. The method may further comprise inserting the plunger into the pharmaceutical container and cooling the pharmaceutical container assembly and the biological or pharmaceutical composition disposed therein to a temperature of less than −65° C. The seal may be maintained when the pharmaceutical container assembly is cooled to less than or equal to −65° C.

A forty-third aspect of the present disclosure may include the method of storing a pharmaceutical composition of the forty-second aspect, where the biological or pharmaceutical composition is added to the pharmaceutical container before the plunger is inserted into the pharmaceutical container.

A forty-fourth aspect of the present disclosure may include the method of storing a pharmaceutical composition of the forty-second aspect, where the biological or pharmaceutical composition is added to the pharmaceutical container after the plunger is inserted into the pharmaceutical container.

It will be apparent to those skilled in the art that various modifications and variations can be made to the embodiments described herein without departing from the spirit and scope of the claimed subject matter. Thus, it is intended that the specification cover the modifications and variations of the various embodiments described herein provided such modification and variations come within the scope of the appended claims and their equivalents.

1-17. (canceled)

18. A pharmaceutical container assembly comprising:
a pharmaceutical container comprising a body having an outer surface and an inner surface; and
a plunger inserted within an opening in the body, wherein:
an outer radial surface of the plunger forms an interference fit with the inner surface of the body;
the plunger comprises a resilient material having a glass transition temperature T_{g} of less than or equal to −65° C.;
the interference fit forms a seal between the inner surface of the body of the pharmaceutical container and the outer surface of the plunger; and
the seal is maintained when the pharmaceutical container assembly is cooled to less than or equal to −65° C., such that a helium leakage rate of the sealed container is less than or equal to 1.4×10−6 cm³/s.

19. (canceled)

20. The pharmaceutical container assembly of claim 18, wherein the interference fit is at least 2%, wherein the interference fit percent is the percent difference between the diameter of the outer radial surface of the plunger and the diameter of the inner surface of the body.

21. The pharmaceutical container assembly of claim 18, wherein the interference fit is at least 5%.

22. The pharmaceutical container assembly of claim 18, wherein the plunger further comprises a biocompatible polymer.

23. The pharmaceutical container assembly of claim 22, wherein the biocompatible polymer comprises one or more of butyl rubber, bromobutyl rubber, chlorobutyl rubber, polytetrafluoroethylene, ethylene tetrafluoroethylene, fluorinated ethylene propylene, perfluoroalkoxy alkane, and copolymers or blends thereof.

24. The pharmaceutical container assembly of claim 22, wherein the biocompatible polymer forms a coating layer on an inner surface of the plunger.

25. The pharmaceutical container assembly of claim 24, wherein the coating layer has a thickness of at least 3 microns.

26. The pharmaceutical container assembly of claim 24, wherein the coating layer has a thickness of from greater than 0% to less than or equal to 75% of the total thickness of the plunger.

27. The pharmaceutical container assembly of claim 25, wherein the coating layer has a thickness of from about 25 microns to about 200 microns.

28. The pharmaceutical container assembly of claim 24, wherein the coating layer is thermoformed onto the plunger.

29. The pharmaceutical container assembly of claim 24, wherein the coating layer is spray coated onto the plunger.

30. The pharmaceutical container assembly of claim 29, wherein the coating is photo −cured after being spray coated onto the plunger.

31. The pharmaceutical container assembly of claim 22, wherein the biocompatible polymer is interspersed within the resilient material.

32. The pharmaceutical container assembly of claim 31, wherein the biocompatible polymer is interspersed within the resilient material on the inner surface of the plunger forming an interspersed polymer layer.

33. The pharmaceutical container assembly of claim 32, wherein the interspersed polymer layer comprises from greater than 0 wt. % to 100 wt. % of the biocompatible polymer based on the total weight of the interspersed polymer layer.

34. The pharmaceutical container assembly of claim 18, wherein the resilient material has a glass transition temperature T_{g} of less than or equal to −80° C.

35. The pharmaceutical container assembly of claim 18, wherein the resilient material has a glass transition temperature T_{g} of greater than or equal to −120° C.

36. The pharmaceutical container assembly of claim 18, wherein the resilient material comprises a silicone rubber that has a glass transition temperature T_{g} of less than or equal to −65° C.

37. The pharmaceutical container assembly of claim 18, wherein the body of the pharmaceutical container comprises glass or polymeric glass.

38. The pharmaceutical container assembly of claim 18, wherein the seal is maintained when the container is cooled to less than or equal to −80° C.

39. (canceled)

40. The pharmaceutical container assembly of claim 18, wherein the pharmaceutical container is a syringe.

41. The pharmaceutical container assembly of claim 18, wherein the pharmaceutical container is a cartridge.

42. A method of storing a pharmaceutical composition, the method comprising:
providing a pharmaceutical container assembly, wherein the pharmaceutical container assembly comprises:
a pharmaceutical container comprising a body having an outer surface and an inner surface; and
a plunger inserted within an opening in the body, wherein:
an outer radial surface of the plunger forms an interference fit with the inner surface of the body;
the interference fit forms a seal between the inner surface of the body of the pharmaceutical container and the outer surface of the plunger; and
the plunger comprises a resilient material having a glass transition temperature of less than or equal to −65° C.;
adding a biological or pharmaceutical composition to the pharmaceutical container;
inserting the plunger into the pharmaceutical container; and
cooling the pharmaceutical container assembly and the biological or pharmaceutical composition disposed therein to a temperature of less than −65° C.,
wherein the seal is maintained when the pharmaceutical container assembly is cooled to less than or equal to −65° C.

43. The method of claim 42, wherein the biological or pharmaceutical composition is added to the pharmaceutical container before the plunger is inserted into the pharmaceutical container.

44. The method of claim 42, wherein the biological or pharmaceutical composition is added to the pharmaceutical container after the plunger is inserted into the pharmaceutical container.
